(19) United States
(12) Patent Application Publication
(10) Pub. No.: US 2025/0283966 A1
(43) Pub. Date: Sep. 11, 2025

(54) SINGLE-SHOT MULTI-B-VALUE AND TIME-DEPENDENT DIFFUSION-WEIGHTED MRI USING SPIN ECHO AND STIMULATED ECHOES WITH VARIABLE FLIP ANGLES

(71) Applicant: THE BOARD OF TRUSTEES OF THE UNIVERSITY OF ILLINOIS, Urbana, IL (US)

(72) Inventors: Xiaohong Joe ZHOU, Naperville, IL (US); Guangyu DAN, Chicago, IL (US)

(21) Appl. No.: 18/856,927

(22) PCT Filed: Apr. 11, 2023
(86) PCT No.: PCT/US2023/018110

§ 371 Date: Oct. 15, 2024

Related U.S. Application Data
(60) Provisional application No. 63/332,262, filed on Apr. 18, 2022.

(51) Int. Cl. G01R 33/561; A61B 5/00; A61B 5/055; G01R 33/563; G01R 33/58

(52) U.S. Cl. CPC G01R 33/5616; A61B 5/0042; A61B 5/055; G01R 33/563; G01R 33/58; A61B 2576/026

(57) ABSTRACT

A time-efficient diffusion magnetic resonance imaging systems and methods are provided that can produce multiple diffusion-weighted images with different b-values in one repetition time, and/or multiple diffusion-weighted images with different diffusion times in one repetition time, both by utilizing a train of stimulated echoes. Each stimulated echo in the train of stimulated echoes is generated by a re-excitation radio-frequency (RE) pulse with a variable flip angle, following an initial 90° excitation RE pulse and a subsequent 90° restoration RE pulse.

### INTRODUCTION

This application claims the benefit of priority from U.S. Provisional Application Ser. No. 63/332,262, filed Apr. 18, 2022, the contents of which are incorporated herein by reference in their entireties.

This invention was made with government support under grant nos. R01EB026716 and 1S10RR028898 awarded by the National Institutes of Health. The government has certain rights in this invention.

### BACKGROUND

With its ability to probe water molecular diffusion at the microscopic scale, diffusion-weighted imaging (DWI) has become a powerful non-invasive technique to characterize microenvironment and infer tissue microstructures. The apparent diffusion coefficient (ADC) quantitatively describes the mobility of water molecules in tissues and has been found useful for characterizing a variety of diseases such as stroke and cancer. In complex biological tissues, it has been increasingly recognized that ADC not only depends on diffusion encoding direction and b-values, but also varies with the diffusion time (A). Diffusion dynamics at different spatial scales can be captured by measuring ADCs at different diffusion times, and the dependence of ADC on A can be used to estimate tissue physical or physiological parameters, such as cell size, surface-to-volume ratio, and intercellular volume fraction. Furthermore, diffusion-time dependency of ADC can also be applied to detecting breast cancer, evaluating head and neck tumors, and grading prostate cancer. As such, diffusion-time dependency can serve as an important biomarker.

Diffusion-weighted images are typically acquired using a single-shot spin-echo echo-planar imaging (SE-EPI) sequence due to its robustness against motion and high time efficiency. Stimulated-echo acquisition mode (STEAM) has also been used to support a long Δ during the mixing time (TM) without excessive signal loss due to T2 decay. Either SE-EPI or STEAM acquires only one diffusion image per repetition time (TR). The sequence must be repeated in DWI studies requiring multiple b-values, leading to long acquisition times, patient compliance issues, and motion-induced image misregistration.

Single-shot spin-echo echo-planar imaging with a preparatory diffusion-weighting module (SE-EPI-DWI) is the dominant DWI pulse sequence in clinical practice and research studies. However, SE-EPI-DWI's ability to study diffusion-time dependency is limited because it cannot incorporate a broad range of diffusion times due to the substantial T2-induced signal loss as Δ increases. To overcome this limitation, STEAM has been proposed to enable a broad range of diffusion times during the TM, when the magnetization is restored along the longitudinal axis, thus avoiding T2 decay while experiencing only a minor to moderate T1 effect.

Common to all time-dependent diffusion magnetic resonance imaging (MRI) studies, the total scan time is a practical constraint because each sequence execution (i.e., each TR) typically corresponds to only a single diffusion time and multiple diffusion times must be employed to investigate the diffusion-time dependency. The lengthy acquisition times not only present a challenge for patient compliance and comfort, but also lead to inaccuracy in quantitative diffusion analysis due to motion-induced image misregistration. Therefore, simultaneous acquisitions with multiple diffusion times in a single sequence are highly desirable to reduce the scan times, improve patient compliance, and increase data reliability. The present invention meets this need in the art.

### SUMMARY OF THE INVENTION

This invention provides a magnetic resonance imaging system composed of a pulse sequence including a stimulated echo configured to incorporate a set of multiple b-values and a set of readouts to generate diffusion-weighted images, each corresponding to a b-value in the set of multiple b-values, within a single scan. In one aspect, the pulse sequence includes a diffusion-weighted preparation module, a spin echo echo-planar-imaging (EPI) module, and a variable flip-angle stimulated echo EPI module. In another aspect, the stimulated echo of the pulse sequence includes an initial 90° excitation radiofrequency (RF) pulse and a subsequent 90° excitation RF, followed by a train of re-excitation RF pulses with variable flip angles (αᵢ), each producing a stimulated echo signal that provides a diffusion-weighted image with its distinctively assigned b-value. In a further aspect, one or more crusher gradients or spoiler gradients are used to select a stimulated echo signal corresponding to a specific and desired b-value. In still another aspect, the variable flip angles (αᵢ) of the train of re-excitation RF pulses are determined by: αᵢ=arctan(sin αᵢ₊₁), where i=2, . . . n, and n is a total number of re-excitation RF pulses in the train of re-excitation RF pulses. As used herein, αₙ₊₁ is set to 90°, hence the index for i is upper-bounded by n, although the flip angles span from α₂ to αₙ₊₁. In some aspects, the magnetic resonance imaging system further includes a calibration procedure to produce quantitative diffusion coefficient maps by reducing or eliminating the perturbations from relaxation times. In a certain aspect, the pulse sequence generates datasets from data acquired in a single shot.

This invention also provides a magnetic resonance imaging system composed of a pulse sequence including a stimulated echo configured to incorporate a set of multiple diffusion times, each corresponding to one diffusion-weighted image in a set of diffusion-weighted images that are collectively acquired in a single scan; the pulse sequence is repeated a plurality of times, each with a unique b-value from a set of b-values. In one aspect, the pulse sequence includes a diffusion-weighted preparation module, a spin echo echo-planar-imaging (EPI) module, and a variable flip-angle stimulated echo EPI module. In another aspect, the stimulated echo of the pulse sequence includes an initial 90° excitation radiofrequency (RF) pulse and a subsequent 90° excitation RF, followed by a train of re-excitation RF pulses with variable flip angles, each producing a stimulated echo signal that provides a diffusion-weighted image with its distinctively assigned b-value. In a further aspect, one or more crusher gradients or spoiler gradients are used to select a stimulated signal corresponding to a specific diffusion time. In yet another aspect, the diffusion-weighted images with the set of multiple diffusion times and the set of b-values provide the influence of diffusion time on quantification of diffusion-related parameters. In a certain aspect, the pulse sequence generates datasets from data acquired in a single shot.

The invention further provides a pulse sequence configured to generate a magnetic resonance image output dataset via a processor, the output dataset including diffusion-weighting with multiple b-values and temporal diffusion characteristics, the pulse sequence comprising multiple stimulated echoes with variable flip angles. In a certain aspect, the pulse sequence is configured to obtain the output dataset from a single shot.

### BRIEF DESCRIPTION OF THE DRAWINGS

FIG. 1 shows a diagram of the single-shot multi-b-value (SSMb) sequence. n+1 EPI images with different diffusion weightings are acquired in a single shot. The sequence contains a diffusion-weighted (DW)-prep module, a spin-echo echo-planar imaging (SEEPI) module, and a variable flip angle stimulated-echo echo-planar imaging (VFA-STEEPI) module. After the 90°_{-X} radiofrequency (RF) pulse in the DW-prep module (where the subscript -X indicates the axis along which the RF pulse is applied.), half of the magnetization remains in the transverse plane to form a spin echo at ET₁ (SEEPI module). The other half is stored along the longitudinal axis, which is repeatedly excited by n consecutive RF pulses (RFᵢ) with variable flip angles, each followed by a second lobe of the diffusion-weighting gradient and an EPI readout (VFA-STEPI module). The spoiler gradient G_{SP} spoils the transverse magnetization after each EPI readout. The amplitude of G_{SP} is set differently to effectively prevent the formation of unwanted spin echoes. The crusher gradient G_{C} and diffusion-sensitizing gradient G_{D} conjointly dephase and rephase the prepared magnetization while spoiling the residual longitudinal magnetization from the unwanted pathway that regrows during TM. G_{SS}, slice selection gradient; ET, echo train readouts; Δᵢ, separation between the dephasing diffusion gradient and the i^{th} rephasing diffusion gradient.

FIG. 2 shows ADC maps of the National Institute of Standards and Technology (NIST) diffusion phantom estimated from the diffusion-weighted images acquired using a commerical SE-EPI sequence and the SSMb sequence, as indicated. The plot shows the correlation of mean ADC values in the 13 phantom compartments from the SE-EPI and SSMb sequences. The ADCs were measured within an ROI of 1.98 cm² in each compartment. The diagonal line serves as a reference representing equal measurements. The vertical bars indicate the errors. The Pearson correlation coefficient was r=0.999.

FIG. 3 provides a set of representative axial diffusion-weighted images from the brain of a healthy human subject (male, 34-year-old), acquired with the SSMb sequence in one shot (4 s). The four upper images correspond to the SEEPI readout (b=16 s/mm²) and the 3 subsequent VFA-STEEPI readouts (b=195 s/mm², b=364 s/mm², and b=533 s/mm²). The bottom row of images correspond to the diffusion-weighted images acquired with the conventional SE-EPI sequence in four separate shots (16 s). Diffusion gradients were applied along the right/left direction in both sequences.

FIG. 4 shows six contiguous axial diffusion-weighted images of the brain acquired with the SSMb sequence within one TR (4 s). The b-values are indicated on each sub-figure. The image for b=16 s/mm² was acquired with ET₁ in the SEEPI module; and the images for b=195 s/mm², b=364 s/mm², and b=533 s/mm² were acquired by the stimulated echoes in the VFA-STEEPI module. Diffusion gradients were applied along the right/left direction.

FIG. 5 shows a set of representative diffusion-weighted axial images from the brain acquired by using the SSMb sequence from a healthy human subject (male, 25-year-old) (upper images). The lower images are the corresponding D_{diff}, D_{perf}, and f parametric maps computed from the images in the top row using an intra-voxel incoherent motion (IVIM) model.

FIG. 6 shows a set of axial prostate diffusion-weighted images (healthy male, 25-year-old) acquired by using the SSMb sequence in one shot. The four images in the upper row were acquired by the SEEPI readout (b=16 s/mm²) and the 3 subsequent VFA-STEEPI readouts (b=195 s/mm², b=364 s/mm², and b=533 s/mm²). The bottom row of images correspond to the diffusion-weighted images acquired by using the conventional SE-EPI sequence in four separate shots with b-values matching those in the upper row. Diffusion gradients were applied along the right/left direction in both sequences.

FIG. 7 shows a diagram of the diffusion-weighted multiple stimulated echoes with variable flip angles (DW-mSTE-VFA) sequence. n echo-planar images, each corresponding to a different diffusion time Δᵢ, are acquired in a single shot. The sequence contains a DW-STE preparation module (left dashed box) and a mSTE-VFA module (right dashed box). In the DW-STE preparation module, the 90°_{X} RF pulse excites the magnetization (where the subscript indicates the phase of the 90° RF pulse). The subsequent 90°_{-X} RF pulse, applied with an opposite phase, restores one half of the magnetization onto the longitudinal axis after it has experienced the first half of the Stejskal-Tanner diffusion gradient pair G_{D} and the left crusher gradient G_{C}, while leaving the other half in the transverse plane that is dephased by the spoiler gradient (G_{TM}) applied during the mixing time TM. After TM, the stored magnetization is successively excited by n RF pulses (RFᵢ) in the mSTE-VFA module. Each RF pulse with a variable flip angle is followed by the second half of the Stejskal-Tanner diffusion gradient pair and the right crusher (G_{C}) prior to the EPI readout. After each acquisition, a spoiler gradient G_{SP} is applied along all three gradient axes to completely spoil the residual transverse magnetization. G_{C}, crusher gradient; G_{SS}, slice selection gradient; ETᵢ, the i^{th} echo train; Δᵢ, separation between the dephasing diffusion gradient lobe (i.e., the first Stejskal-Tanner diffusion gradient lobe) and the i^{th} rephasing diffusion gradient lobe (i.e., the second Stejskal-Tanner diffusion gradient lobe).

FIG. 8 shows ADC maps of the NIST diffusion phantom estimated from the diffusion-weighted images acquired by using ET₁, ET₂, and ET₃ of the DW-mSTE-VFA sequence and the commercial SE-EPI-DWI sequence, as indicated. The plots show the mean ADC values measured in 13 different compartments of the phantom by DW-mSTE-VFA at ET₁, ET₂, and ET₃, vs. those by SE-EPI-DWI. The ADCs were averaged within a region of interest (ROI) of 1.98 cm² in each compartment. The diagonal line serves as a reference representing equal measurements. The vertical bars indicate the standard deviation of the voxel-wise ADC values within each ROI in the DW-mSTE-VFA measurement.

FIG. 9 provides a set of representative mango ADC maps calculated from DW images acquired with the DW-mSTE-VFA (upper row) and standard DW-STE (lower row) sequences. A and b-values in the DW-STE acquisition matched those in the DW-mSTE-VFA acquisition.

FIG. 10A shows a set of representative brain ADC maps estimated by using DW images acquired at ET₁, ET₂, and ET₃ of the DW-mSTE-VFA sequence, respectively.

FIG. 10B shows box plots of the mean ADC values computed over the ROIs in the white matter (anterior corona radiata) and gray matter (putamen), as indicated. The p-value generated by Friedman's test is shown in the bottom right corner of each box plot. The p-values generated by post-hoc paired t-test are indicated on the top of each box plot. * indicates statistically significant difference using p<0.05 as a threshold.

FIG. 11 shows a set of representative brain ADC maps calculated from DW images acquired at ET₂, ET₂, and ET₃ of the DW-mSTE-VFA sequence, as indicated (upper images). The mean ADC values of white matter (WM) and gray matter (GM) in the upper images at ET₁, ET₂, and ET₃ were 0.751/0.798, 0.693/0.683, and 0.656/0.621 μm²/ms, respectively. The lower images are a set of brain ADC maps calculated from the DW images acquired using the commercial SE-EPI-DWI sequence with b_{ET1}, b_{ET2}, and b_{ET3}, as indicated, which matched to those used in the DW-mSTE-VFA sequence. The mean ADC values of WM and GM in the lower images with b_{ET1}, b_{ET2}, and b_{ET3} were 0.839/0.874, 0.783/0.763, and 0.758/0.747 μm²/ms, respectively. Each column corresponds to the same slice from the subject.

FIG. 12 shows a set of representative brain ADC maps calculated from DW images acquired using the DW-mSTE-VFA (upper row) and standard DW-STE sequences (lower row) from the same subject. Δ and b-values in the DW-STE acquisition matched those in the DW-mSTE-VFA acquisition. Each column corresponds to the same slice from the subject.

FIG. 13A shows a set of representative prostate ADC maps estimated by using DW images acquired at ET₁, ET₂, and ET₃ of the DW-mSTE-VFA sequence, respectively.

FIG. 13B shows box plots of the mean ADC values computed over the ROIs in the peripheral zone and central gland, as indicated. The p-value generated by Friedman's test is shown in the bottom right corner of each box plot. The p-values generated by post-hoc paired t-test are indicated on the top of each box plot. * indicates statistically significant difference using p<0.05 as a threshold.

### DETAILED DESCRIPTION OF THE INVENTION

The present invention provides systems and methods to acquire multiple diffusion-weighted magnetic resonance images with different b-values in a single shot. In one aspect, the system or method is referred to as “Spin Echo and Stimulated Echoes with Variable Flip Angles (SESTE-VFA)” acquisition. SESTE-VFA (also referred to herein as SSMb) was implemented based on a diffusion-weighted stimulated-echo EPI sequence. After the leading pair of 90° RF pulses and an in-between diffusion gradient lobe (GD), half of the magnetization is stored along the longitudinal axis, which is repeatedly re-excited by a series of (n) low-flip-angle pulses, each followed by a GD and an EPI readout train to sample the magnetization at or near the stimulated echo, resulting in a total of n stimulated-echo echo-train acquisitions. The other half of the magnetization remains in the transverse plane, which is rephased by the second lobe of GD to form a spin echo that is acquired by the first echo-train readout. As such, (n+1) diffusion images with different b-values are acquired in a single shot. SESTE-VFA not only enables single-shot multi-b-value diffusion MRI acquisition, but also provides an alternative approach to vary b-value by fixing diffusion gradient amplitude while varying diffusion time. Quantitative diffusion time-dependent analysis can also be incorporated with SESTE-VFA to investigate the diffusion dynamics at different temporal scales, which can be further used to estimate tissue physical or physiological parameters. Utility of the systems and methods has been demonstrated in a diffusion phantom, human brain, and prostate and been shown to reduce the scan times by at least four-fold. Accordingly, the systems and methods can be used for brain imaging, prostate imaging, pelvis imaging, abdomen imaging, etc., all of which can benefit clinical and research use of MRI on human subjects and animals. Advantageously, the systems and methods can be applied in both Gaussian and non-Gaussian model-based diffusion imaging studies, can improve patient compliance, and facilitate diffusion-weighted image co-registration.

Diffusion-weighted imaging, among other magnetic resonance imaging (“MRI”) techniques, can provide invaluable information about the structure and function of various tissues in the body. In a diffusion-weighted pulse sequence, a pair of diffusion encoding gradients, or gradient waveforms, are typically applied along a direction to attenuate the transversal magnetization in a volume of tissue. The detected signal intensity depends on the diffusion of water in tissues. The “b-value” of a diffusion-weighted pulse sequence (measured in units of s/mm²) indicates the degree of diffusion-weighting in an acquired image and dictates the level of signal attenuation as a function of tissue diffusivity. The b-value is determined in general by the strength and duration of the applied gradients and in some cases by the time interval between applied gradients. Higher b-values increase the effect of diffusion on the signal and decrease the overall signal intensity.

The systems and methods of this invention find application in the diagnosis and treatment of acute brain ischemia, brain tumors, white matter diseases, pediatric brain development and aging, oncological applications (brain, head and neck, breast, prostate, liver, hepatobiliary and pancreatic cancers), bowel disorders, genito-urinary applications, peripheral nerve imaging, and musculoskeletal applications.

### Single-Shot Multi-b-Value (SSMb) Diffusion Imaging.

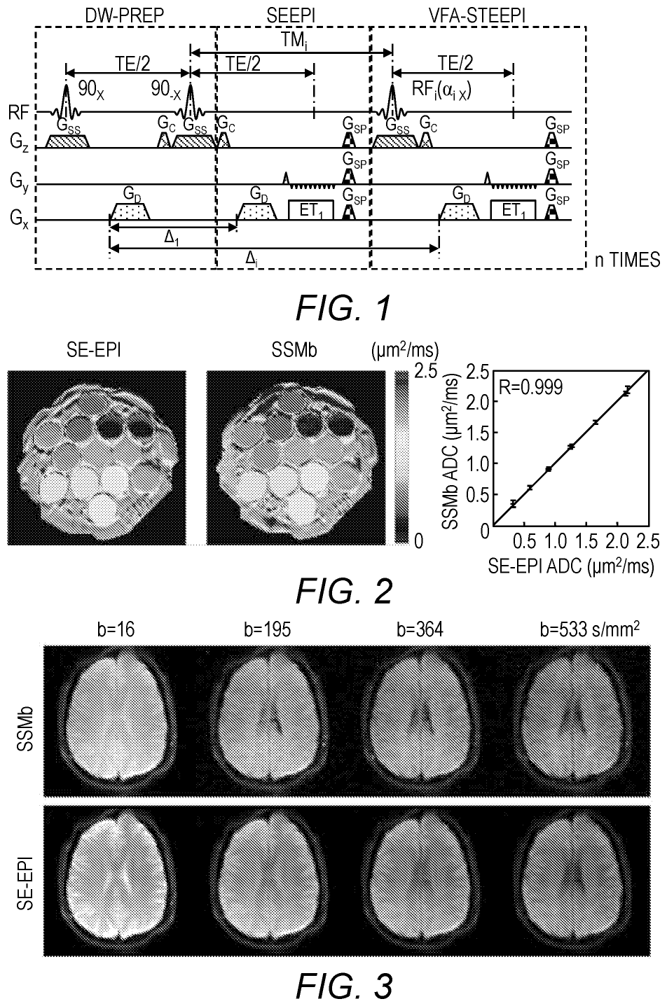

Pulse Sequence Design. The SSMb sequence (FIG. 1) can be composed of three modules: a diffusion-weighted preparation (DW-prep) module, a spin-echo echo-planar imaging (SEEPI) module, and a variable flip angle stimulated-echo echo-planar imaging (VFA-STEEPI) module. The DW-prep module (inside the left dashed box in FIG. 1) contains a pair of 90° radiofrequency (RF) pulses to produce and restore half of the magnetization onto the longitudinal axis, while the other half remains in the transverse plane. In the SEEPI module (as shown inside the center dashed box of FIG. 1), a diffusion-weighting gradient G_{D} and a crusher gradient G_{C} can be applied. They can work in unison with their corresponding counterpart in the first half of the echo time (TE)/2 during the DW-prep module to rephase the transverse magnetization to form a spin echo which is acquired by the first echo-train readout (ET₁). For the stored longitudinal magnetization, instead of using another 90° RF pulse for re-excitation as in a conventional STEAM sequence, the VFA-STEEPI module (as shown inside the right dashed box in FIG. 1) is employed with n low-flip-angle RF pulses, each followed by a rephasing diffusion gradient lobe G_{D} as well as a complementary crusher gradient lobe Go. After each low-flip-angle RF pulse, an EPI-like readout train (ETᵢ) is applied to acquire the signals during the formation of the stimulated echo, resulting in n echo-train acquisitions all having the same TE. These stimulated echoes, together with the preceding spin-echo acquired in the SEEPI module, produces a total of (n+1) signals that are spatially encoded to form (n+1) images following each shot (or TR).

Signal Characteristics. Assuming TR>>T1, 90° flip angles for the initial two RF pulses in FIG. 1, and mono-exponential T1-, T2-, and diffusion-induced signal decay (or recovery), the signal characteristics of the echo train in the SEEPI module (S_{ET}_{₁}) and the VFA-STEEPI module (S_{ET}_{ᵢ}) are given as:

$${S}_{E {T}_{1}} = \frac{1}{2} {M}_{z}^{0} ⋆ exp ( − {b}_{1} ⋆ D ) ⋆ exp ( − \frac{TE}{T 2} ) , \qquad Equation [ 1 ]$$

$$Equation [ 2 ]$$

$${S}_{E {T}_{i}} = \{ \begin{matrix}\frac{1}{2} {M}_{z}^{0} sin ( {α}_{i} ) exp ( − \frac{{TM}_{i}}{T 1} ) ⋆ exp ( − {b}_{i} ⋆ D ) ⋆ exp ( − \frac{TE}{T 2} ) , \text{} i = 2 \\ \frac{1}{2} {M}_{z}^{0} [ {\prod }_{j = 1}^{i − 1} cos ( {α}_{j} ) ] sin ( {α}_{i} ) exp ( − \frac{{TM}_{i}}{T 1} ) ⋆ exp ( − {b}_{i} ⋆ D ) ⋆ exp ( − \frac{TE}{T 2} ) , i > 2\end{matrix}$$

where M_{z}⁰ is the bulk magnetization at thermal equilibrium; TMᵢ is the time interval between the second 90° RF pulse and the RFᵢ pulse in the VFA-STEEPI module; b₁ is the effective b-value for ET₁ in the SEEPI module; bᵢ is the effective b-value for ETᵢ in the VFA-STEEPI module (i=2, 3, . . . , n+1). These b-values are given by:

$${b}_{1} = {γ}^{2} {\int }_{0}^{TE} {[ {\int }_{0}^{t} {P}_{SEEPI} * {G}_{D} ( {t}^{′} ) {dt}^{′} ]}^{2} dt , \qquad Equation [ 3 ]$$

$${b}_{i} = {γ}^{2} {\int }_{0}^{TE + {TM}_{i}} {[ {\int }_{0}^{t} {P}_{VFA - STEEPI} * {G}_{D} ( {t}^{′} ) {dt}^{′} ]}^{2} d t , \qquad Equation [ 4 ]$$

where γ is the gyrometric ratio, G_{D} is the diffusion gradient amplitude, and polarity P is given by:

$${P}_{SEEPI} = \{ \begin{matrix}1 , & 0 < {t}^{′} \leq TE / 2 \\ − 1 , & \frac{TE}{2} < {t}^{′} < TE\end{matrix} \qquad Equation [ 5 ]$$

$${P}_{V F A − STEEPI} = \{ \begin{matrix}1 , & 0 < {t}^{′} \leq \frac{TE}{2} \\ 0 , & \frac{TE}{2} < {t}^{′} \leq \frac{TE}{2} + T {M}_{i} \\ − 1 , & \frac{TE}{2} + T {M}_{i} < {t}^{′} < T E + T {M}_{i}\end{matrix} \qquad Equation [ 6 ]$$

Calibrations for Multiple b-Values in a Single-Shot Acquisition. According to Eqs. [1] and [2], the signal at each echo train is determined by the compounded effect of the previous RF pulses with low flip angles, T1, T2, and diffusion coefficient. Different degrees of diffusion weighting (or b-values) among the different echo trains are introduced by the different diffusion time Δᵢ. This provides the possibility of acquiring a set of DW images with multiple b-values in a single shot. However, the echo trains in the VFA-STEEPI module have different T1-weighting due to the varying TMs and flip angles, leading to signal fluctuations in the DW images unrelated to diffusion weighting. To compensate for this effect, a calibration procedure was implemented.

The calibration was composed of a baseline acquisition using the SSMb sequence without diffusion-weighting gradients. The other imaging parameters were kept the same as the nominal DW image acquisition. The signals at the i^{th} echo train in the baseline (S_{ET}_{ᵢ}_{,basline}) and nominal (S_{ET}_{ᵢ}_{,nominal}) acquisitions in Eqs. [1] and [2] can be re-written as:

$${S}_{E {T}_{i} , baseline} = {A}_{i} exp ( − {b}_{i , baseline} ⋆ D ) , \qquad Equation [ 7 ]$$

$${S}_{E {T}_{i} , nominal} = {A}_{i} exp ( − {b}_{i , nominal} ⋆ D ) , \qquad Equation [ 8 ]$$

$$where i = 1 , 2 , … … n + 1 , and$$

$$Equation [ 9 ]$$

$${A}_{i} = \{ \text{} \begin{matrix}\frac{1}{2} {M}_{z}^{0} \cdot exp ( − \frac{TE}{T 2} ) , & i = 1 ( SEEPI ) \\ \frac{1}{2} {M}_{z}^{0} sin ( {α}_{i} ) exp ( − \frac{{TM}_{i}}{T 1} ) ⋆ ex ( − \frac{TE}{T 2} ) , & i = 2 ( VFA − STEEPI ) \\ \frac{1}{2} {M}_{z}^{0} [ {\prod }_{j = 2}^{i − 1} cos ( {α}_{j} ) ] sin ( {α}_{i} ) exp ( − \frac{{TM}_{i}}{T 1} ) ⋆ exp ( − \frac{TE}{T 2} ) , & i > 2 ( VFA − STEEPI )\end{matrix}$$

Relative to the baseline image, the diffusion-induced signal attenuation (Iᵢ) was quantified by dividing Eq. [8] with Eq. [7]:

$$Equation [ 10 ]$$

$${I}_{i} = \frac{{S}_{E {T}_{i} , nominal}}{{S}_{E {T}_{i} , baseline}} = \frac{{A}_{i} exp ( - {b}_{i , nominal} * D )}{{A}_{i} exp ( - {b}_{i , baseline} * D )} = exp - ( {b}_{i , nominal} - {b}_{i , baseline} ) ⋆ D ) .$$

Finally, n+1 calibrated DW images were obtained by multiplying Iᵢ with S_{ET}_{₁}_{,baseline}

$$\begin{matrix}{S}_{i} = {I}_{i} * {S}_{E {T}_{1} , b a s e l i n e} \\ = \begin{matrix}\frac{1}{2} {M}_{z}^{0} exp ( - \frac{TE}{T 2} ) * exp ( - \\ ( {b}_{i , nominal} - {b}_{i , baseline} + {b}_{1 , baseline} ) * D ) .\end{matrix}\end{matrix} \qquad Equation [ 11 ]$$

Sᵢ represents the signal intensity in the i^{th} calibrated DW image in which the effective b-factor is given by b_{i,nominal}−b_{i,baseline}+b_{1,baseline}, where b_{i,baseline} is the b-value contributed by imaging gradients in the i^{th} echo train. It can be seen that Sᵢ is modulated by only the diffusion weighting factor exp(−(b_{i,nominal}−b_{i,baseline}+b_{1,baseline})*D) and T2 decay while being independent of TMᵢ and flip angles of previous RF pulses. If b_{1,baseline} is substantially close to zero, then it can be dropped from Eq. [11].

Flip Angle Optimization. Determination of the flip angles αᵢ is crucial to the SSMb sequence. If a constant small flip angle αᵢ is used to produce all stimulated echoes, substantial longitudinal magnetization may remain at the end of the last echo train and is therefore wasteful. Conversely, if a constant large flip angle is used, the first few echo trains will consume much of the stored magnetization, resulting in a low SNR for the later echo trains. To balance these two scenarios, a variable flip angle approach can be employed, in which the individual flip angles αᵢ are calculated recursively. Ignoring T1 and diffusion induced signal attenuation, starting with αₙ₊₁=90° (i.e., the last echo train consumes all available magnetization), the flip angles of the preceding RF pulses are determined from Eq. [12]:

$${α}_{i} = arctan ( sin {α}_{i + 1} ) , \qquad Equation [ 12 ]$$

$$where i = 2 , … … n .$$

Pulse Sequence Implementation. The SSMb sequence was implemented on a GE MR750 3T scanner (GE Healthcare, Waukesha, WI) with a maximum gradient amplitude of 50 mT/m and a maximum gradient slew rate of 200 mT/m/ms. In the experimental demonstrations, n=3 was chosen due to the signal-to-noise ratio (SNR) constraints. According to Eq. [12], α₂, α₃, and α₄ were determined to be 35.3°, 45.0°, and 90.0°, respectively. In all SSMb experiments, calibration acquisitions were incorporated into the nominal acquisitions in a single sequence with the same parameters except for nullified diffusion-weighting gradients (i.e., b_{i,baseline}˜0).

Phantom Validation. A NIST diffusion phantom was used in the experimental study to verify the accuracy of SSMb in ADC measurement. The phantom was composed of 13 compartments with variable polyvinylpyrrolidone (PVP) concentrations in water, resulting in variable ADCs. Axial images were acquired using an 8-channel head coil (Invivo Corp., Gainesville, FL) with the following SSMb sequence parameters: TR=4000 ms, TE=50.7 ms, TM=[100, 200, 300] ms, FOV=20×20 cm², matrix size=128×128, number of slices=6, slice thickness=5 mm, inter-slice gap=1 mm, b-values for each echo train=[37, 395, 733, 1071] s/mm², diffusion gradient direction=right/left, NEX=8, and scan time=32 s. For comparison, reference DW images were also acquired with the same b-values by using a commercial single-shot SE-EPI sequence (TE=65.7 ms, scan time=60 s, and the other acquisition parameters matched those of the SSMb sequence). For each pulse sequence, an ADC map was estimated using a mono-exponential model:

$$\frac{S ( b )}{{S}_{0}} = exp ( - b * A D C ) \qquad Equation [ 13 ]$$

where S(b) is the signal intensity at b, and S₀ is the signal intensity without diffusion weighting. An iterative Levenberg-Marquardt algorithm was employed in the voxel-wise fitting using all available b-values on the MATLAB platform (MathWorks, Inc, Natick, MA).

A circular region of interest (ROI) with an area of 1.98 cm² (81 voxels) was placed at the center of each compartment in the phantom. Measurements were made by calculating the mean and standard deviation of the ADC within each ROI. To evaluate the agreement between ADCs obtained from SSMb and the commercial single-shot SE-EPI sequence, Pearson correlation coefficient was used for evaluation.

In vivo Experiment on Healthy Human Brain. With approval from the Institutional Review Board (IRB) and written informed consent, the brains of healthy human subjects were scanned with an 8-channel head coil (Invivo Corp., Gainesville, FL) to demonstrate the acquisition efficiency of SSMb and its accuracy and compatibility in mapping diffusion parameters with Gaussian and non-Gaussian diffusion models.

The following acquisition parameters were employed to illustrate the capability of SSMb in scan time reduction and the accuracy of estimating ADC from SSMb images using the Gaussian diffusion model (Eq. [13]): TR=4000 ms, TE=40 ms, TM=[100, 200, 300] ms, FOV=20×20 cm², matrix size=96×96, number of slices=6, slice thickness=7 mm, inter-slice gap=0 mm, b-values at each echo train=[16, 195, 364, 533] s/mm², diffusion gradient direction=right/left, NEX=1, scan time=4 s (i.e., one TR for four b-values). For comparison, images using a commercial single-shot SE-EPI sequence were also acquired with the same b-values, TR/TE=4000 ms/65.7 ms, and the scan time=16 s (i.e., one TR for each b-value). ADC maps were produced by using the same fitting procedure as in the phantom experiment. Histograms of voxel-wise ADC values covering the entire selected slice were plotted to evaluate the agreement of ADC measurements between SSMb and single-shot SE-EPI.

To illustrate the compatibility of SSMb with advanced non-Gaussian diffusion models, an intravoxel incoherent motion (IVIM) diffusion experiment was performed with a similar protocol to the ADC mapping experiment, except for TE=40.9 ms, matrix size=96×96, slice thickness=7 mm, b-values=[24, 295, 551, 807] s/mm², NEX=32 to increase the SNR, and scan time=2 min and 8 s. The images were analyzed using Eq. [14]:

$$\frac{S ( b )}{{S}_{0}} = f exp ( - b {D}_{perf} ) + ( 1 - f ) exp ( - b {D}_{diff} ) \qquad Equation [ 14 ]$$

where D_{diff} is diffusion coefficient, D_{perf} is pseudo-perfusion coefficient, and f is pseudo-perfusion fraction. The three IVIM parameters were estimated voxel-by-voxel by fitting Eq. [14] to the diffusion images acquired with the SSMb sequence by using established methods (Qi et al. (2018) Eur. Radiol. 28:1301-1309).

Prostate Imaging. With IRB approval and written informed consent, prostate images were acquired on healthy human subjects using the SSMb sequence with a 32-channel phased-array pelvis coil. The acquisition parameters were: TR=4000 ms, TE=56.5 ms, FOV=20×20 cm², slice thickness=5 mm, number of slices=6, inter-slice gap=0 mm, TM=[60, 125, 200] ms, and b-values=[49, 295, 534, 810] s/mm², matrix size=128×128, NEX=2, and scan time=8 s. For comparison, the corresponding DW images were acquired using a commercial single-shot SE-EPI sequence with the same acquisition parameters as in the SSMb scan except for TE=62.8 ms, and scan time=16 s. ADC maps were generated using the similar methods described above for the phantom and human brain scans.

Results of Phantom Validation. FIG. 2 shows the ADC maps of the diffusion phantom, from images acquired with the single-shot SE-EPI and SSMb sequences, respectively. In FIG. 2, the ADCs measured by SSMb from each phantom compartment were plotted against the ADCs measured by single-shot SE-EPI. A high Pearson correlation coefficient (r=0.999) was observed, together with a low intra-ROI standard deviation (standard deviation/mean value<1.6% for all compartments). The excellent agreement between the SSMb and single-shot SE-EPI measurements indicates that the more time-efficient SSMb sequence can replace single-shot SE-EPI for ADC quantification.

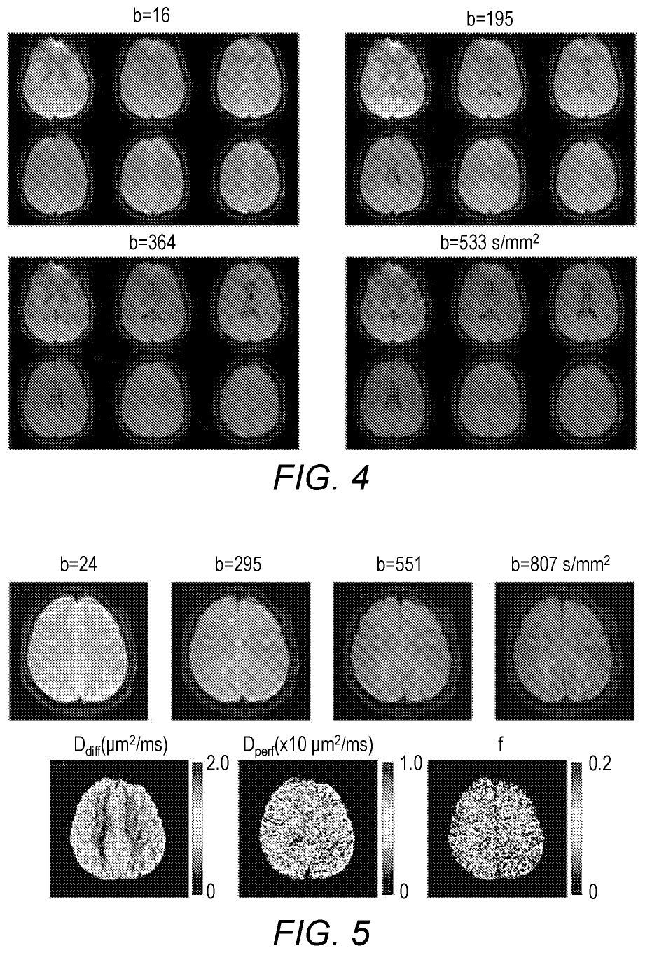

Brain Imaging Results. FIG. 3 displays a set of DW brain images acquired using a single-shot SSMb sequence with four b-values in 4 s (top row), together with the corresponding SE-EPI images (bottom row) that were acquired four times longer (16 s). The SSMb and SE-EPI images showed the same signal attenuation as the b-value increased. As expected, the SSMb images exhibited a lower SNR than the SE-EPI images (165 vs. 323, 69 vs. 289, 60 vs. 280, and 58 vs. 248 at b-value=16, 195, 364, and 533 s/mm², respectively) primarily due to the use of stimulated echoes. However, the SNR loss (˜49% in SEEPI module, ˜76% in VFA-STEEPI module) was offset by a 4-fold scan time reduction (from 16 s to 4 s). FIG. 4 illustrates the multi-slice imaging capability of the SSMb sequence where multiple slices were acquired in a single TR using an interleaved mode.

Representative brain ADC maps from a randomly selected slice acquired with the conventional SE-EPI and SSMb sequences were obtained. Their corresponding histograms were also generated. The histogram from the SSMb sequence exhibited 85.3% overlap with that obtained from the conventional SE-EPI scan. The average ADC values obtained from the SSMb (average ADC=1.02±0.71 μm²/ms) were about 11% lower than the average ADC value estimated by using SE-EPI (average ADC=1.14±0.68 μm²/ms) images.

FIG. 5 displays a set of brain DW images acquired in the IVIM diffusion experiment using the SSMb sequence with four b-values. The bottom row of FIG. 5 shows the IVIM parameter maps computed from the images in the top row, illustrating the compatibility of SSMb with IVIM diffusion analysis by acquiring all raw DW images in a single shot.

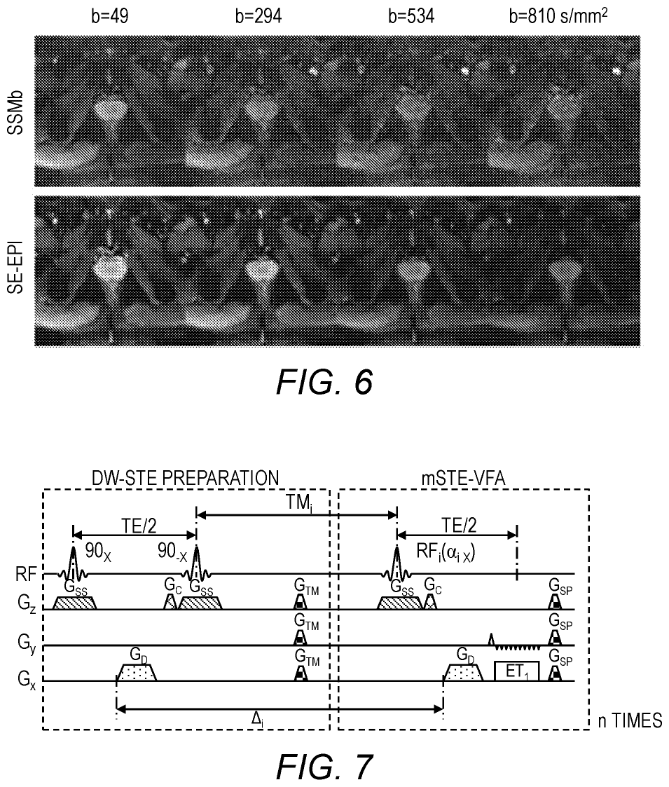

Prostate Imaging Results. An example of SSMb's application to an organ other than the brain is demonstrated in FIG. 6, where a set of prostate DW images acquired using the SSMb sequence with four b-values in a single shot is shown. For comparison, the corresponding images using the conventional SE-EPI sequence with four separate acquisitions (one for each b-value) are also displayed in the bottom row of FIG. 6. The increased acquisition efficiency was at the expense of reduced SNR, the SNR in the central zone of the prostate decreased from [54, 39, 31, 23] in the SE-EPI images to [38, 20, 17, 15] in the SSMb images at b-values=[49, 294, 534, 810] s/mm², respectively. However, the SSMb images provided sufficient SNR for mapping ADC values in the prostate, which were lower than those estimated from the SE-EPI scans. This finding was consistent with that in the brain imaging.

Diffusion-Weighted Multiple Stimulated Echoes with Variable Flip Angles (DW-mSTE-VFA) for Studying Diffusion-Time Dependency.

Pulse Sequence Design. The DW-mSTE-VFA sequence (FIG. 7) is composed of a diffusion-weighted stimulated-echo (DW-STE) preparation module and another module for multi-stimulated echo acquisitions whose re-excitation RF pulses have variable flip angles (i.e., the mSTE-VFA module). In the DW-STE preparation module (as shown inside the left dashed box in FIG. 7), the 90°_{X} RF pulse excites the magnetization (note that the subscript X denotes the phase of the RF pulse), followed by the first half of a Stejskal-Tanner gradient pair (G_{D}) and a left crusher gradient (G_{C}) that jointly introduce diffusion weighting and dephase the transverse magnetization. The subsequent 90°_{-X} RF pulse, applied with an opposite phase to the phase of the initial 90% RF pulse, restores one half of the magnetization onto the longitudinal axis, while leaving the other half in the transverse plane, which is spoiled by a set of spoiler gradients (G_{TM}) during the mixing time TM. The stored longitudinal magnetization experiences T1 relaxation and serves as the signal source for the subsequent mSTE-VFA module. Instead of using another 90°_{X} RF pulse for re-excitation as in a conventional STEAM sequence, the mSTE-VFA module (as shown inside the right dashed box in FIG. 7) employs a series of (n) low-flip-angle pulses to successively re-excite the stored longitudinal magnetization, each followed by the second half of a Stejskal-Tanner gradient pair G_{D} and a complementary right crusher gradient lobe G_{C}. These two gradients work in unison with their respective counterparts in the initial TE/2 period to refocus the magnetization, thus forming a series of stimulated echoes, all having the same TE but different Δ. Each of the resulting multiple stimulated echoes is acquired with an EPI echo train (ETᵢ, where i is the echo-train index) that corresponds to a distinctive diffusion time Δᵢ. As such, the train of multiple stimulated echoes produces a set of DW images with varying diffusion times in a single shot. After each EPI echo-train, a spoiler gradient G_{SP} is applied along all three gradient axes to completely spoil the transverse magnetization without interfering with the diffusion weighting of the subsequent echo trains. In the sequence design, the spoiler gradients produced at least a phase dispersion of 4π within a voxel to prevent formation of unwanted spin echoes.

A comprehensive b-value calculation was used to account for the contributions to the b-value from both imaging and diffusion gradients.

$${b}_{i} = {γ}^{2} {\int }_{0}^{TE + {TM}_{i}} {[ {\int }_{0}^{t} P ( {t}^{′} ) G ( {t}^{′} ) d {t}^{′} ]}^{2} dt , \qquad Equation [ 15 ]$$

where bᵢ and TMᵢ are the effective b-value and TM of the i^{th} mSTE-VFA module, respectively. γ is the gyrometric ratio, G is the gradient amplitude, and the polarity P is given by:

$$P ( {t}^{′} ) = \{ \begin{matrix}1 , & 0 < {t}^{′} \leq \frac{TE}{2} \\ 0 , & \frac{TE}{2} < {t}^{′} \leq \frac{TE}{2} + T {M}_{i} \\ − 1 , & \frac{TE}{2} + T {M}_{i} < {t}^{′} < T E + T {M}_{i}\end{matrix} \qquad Equation [ 16 ]$$

The established variable flip angle approach (Hiepe et al. (2011) Z. Med. Phys. 21:216-227) was employed to equalize the magnetizations across the EPI readout train. Ignoring T1 and diffusion induced signal attenuation and starting with αₙ=90° for the last stimulated echo, the flip angles (αᵢ) of the preceding RF pulses were calculated recursively by:

$${α}_{i} = arctan ( sin {α}_{i + 1} ) ) \qquad Equation [ 17 ]$$

$$where i = 1 , … … n - 1.$$

Data Acquisition and Analysis. The DW-mSTE-VFA sequence was implemented on a GE MR750 3T scanner (GE Healthcare, Waukesha, WI) with a maximum gradient amplitude of 50 mT/m and a maximum gradient slew rate of 200 T/m/s. In the experimental studies, n=3 was chosen due to the SNR constraint at 3T. According to Eq. [17], α₁, α₂, and α₃ were determined to be 35.3°, 45.0°, and 90.0°, respectively. Experiments on a phantom, a mango fruit, and in vivo healthy human brain and prostate were performed to demonstrate the capability of DW-mSTE-VFA for diffusion-time dependency studies.

Phantom Validation. A NIST diffusion phantom was used in the phantom study. This phantom was composed of 13 compartments with variable PVP concentrations in water, resulting in variable diffusion coefficients. Axial images were acquired using an 8-channel head coil (Invivo Corp., Gainesville, FL). In the DW-mSTE-VFA sequence, Δ was set at 119.7, 219.7, and 319.7 ms for ET₁, ET₂, and ETs, respectively. Seven different diffusion gradient amplitudes were applied to produce the following b-values at ET₁/ET₂/ET₃: b_{ET1}/b_{ET2}/b_{ET3} of [0, 100, 200, 300, 400, 500, 1000]/[0, 185, 371, 556, 741, 927, 1853]/[0, 271, 541, 812, 1083, 1353, 2707] s/mm². The other parameters were: TR=4000 ms, TE=50.7 ms, FOV=20×20 cm², matrix size=128×128, slice thickness=5 mm, NEX=8, and scan time=3 min and 48 s. Additionally, a set of reference DW images were acquired with b-values=[0, 100, 200, 300, 400, 500, 1000] s/mm² and Δ=34.8 ms by using a commercial SE-EPI-DWI sequence. ADC maps were estimated from all DW datasets using a mono-exponential model:

$$S ( b ) / {S}_{0} = exp ( - b * A DC ) , \qquad Equation [ 18 ]$$

where S(b) and S₀ are the signal intensities with and without diffusion weighting, respectively. Mean and standard deviation of the ADC values were calculated within a 1.8 cm² circular ROIs around the center of each compartment. Pearson correlation coefficient was used to evaluate the agreement on ADC between measurements from the DW-mSTE-VFA sequence and the commercial SE-EPI-DWI sequence.

Evaluation on a Mango Fruit. The homogeneous phantom experiment above was limited to confirmation of proper sequence implementation. To demonstrate the diffusion-time dependency in a heterogeneous, restricted diffusion medium and validate the performance of the DW-mSTE-VFA sequence, a fresh mango fruit was scanned using the phantom scan protocol described above. For comparison, three sets of DW images were also acquired using a DW stimulated echo echo-planar-imaging (DW-STE) sequence. The Δ and b-values used in the DW-STE sequence were the same as those in the DW-mSTE-VFA sequence for ET₁, ET₂, and ET₃ acquisitions, respectively. At each diffusion time, mean and standard deviation of ADC values from all voxels in the pulp of mango were evaluated, followed by a comparison between the DW-mSTE-VFA and DW-STE sequences by calculating the percentage difference of the measured ADCs.

Brain Imaging. With IRB approval and written informed consent, brain imaging experiments were conducted on six healthy subjects (age range: 31.7±6.9 years) to investigate diffusion-time dependence by using the DW-mSTE-VFA sequence. The same imaging protocol as in the phantom experiment was employed. After the ADC map was obtained for each echo train using a mono-exponential fitting, ROIs were placed on the white matter (WM, 2.0 cm²) in the anterior corona radiata and gray matter (GM, 1.7 cm²) in the putamen, respectively, for all subjects. The average ADC values within each ROI were computed. A Friedman's test was performed to evaluate the overall statistical significance of variations in ADC across the three diffusion times, followed by a post-hoc analysis by using paired t-test to investigate the pairwise differences between diffusion times. p-Values less than 0.05 was selected to indicate statistically significant difference in both the Friedman's test and paired t-test.

On the human brain, two comparisons were performed. First, because different b-values are associated with images acquired at different diffusion times, any potential diffusion-time dependency of ADC must be validated against confounding ADC change caused by b-values. The first comparison was performed between the ADC maps obtained from the DW-mSTE-VFA sequence at the three Δᵢ's described earlier and the ADC maps acquired with a commercial SE-EPI-DWI sequence with b-values of b_{ET2}, b_{ET2}, and b_{ET3}, respectively, at a constant diffusion time of Δ=37.4 ms. Second, to validate the observed diffusion-time dependency, the ADC maps from the DW-mSTE-VFA sequence at the three Δᵢ's were compared with those obtained from the DW-STE sequence with matching Δ and b-values in a similar fashion as described for the mango experiment. The SNR was calculated by using signal intensity in the WM/GM divided by the average of background standard deviations over four regions (each with an area of about 1000 mm²) at the corners of the image.

Prostate Imaging. With the IRB approval and written informed consent, prostate imaging was performed on six healthy subjects (age range: 31.7±6.9 years) using a 32-channel phased-array coil. A similar imaging protocol to those described above was employed with the following key acquisition parameters: Δ=83.3, 148.3, and 223.3 ms for ET₂, ET₂, and ET₃, respectively, corresponding to b-values of [0, 100, 200, 300, 400, 500, 800, 1000] s/mm², [0, 181, 362, 543, 724, 905, 1448, 1810] s/mm², and [0, 275, 549, 824, 1098, 1373, 2196, 2745] s/mm², respectively, TR=4000 ms, TE=56.5 ms, slice thickness=4 mm, NEX=16, and scan time=8 min and 36 s. ROIs were drawn in the peripheral zone (PZ, 1.6 cm²) and central gland (CG, 0.8 cm²) for quantitative ADC analysis. Similar Friedman's test and post-hoc paired t-test to those described for the brain imaging study were performed in the PZ and CG regions.

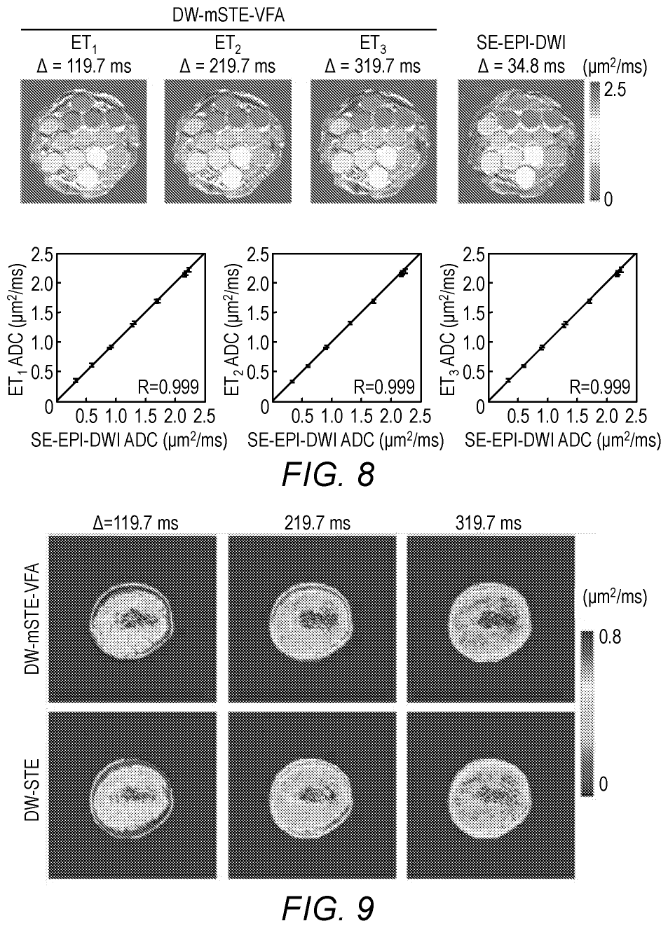

Results of Phantom Validation. FIG. 8 displays the ADC maps of the NIST diffusion phantom generated by ET₁ (Δ=119.7 ms), ET₂ (Δ=219.7 ms), and ET₃ (Δ=319.7 ms) of the DW-mSTE-VFA sequence, respectively, together with the ADC map from the SE-EPI-DWI sequence (Δ=34.8 ms) for comparison. The ADC maps from the DW-mSTE-VFA sequence exhibited comparable image quality and uniform ADC values within each compartment. In FIG. 8, mean ADC values obtained from each DW-mSTE-VFA echo train were plotted against the corresponding mean ADC values from the commercial SE-EPI-DWI sequence using the ROI data from each of the 13 compartments. The mean ADC values measured at different Δ's were similar and highly correlated (r=0.999) with the measurement from the SE-EPI-DWI sequence (Δ=34.8 ms) for all diffusion times, as expected from uniform diffusion media in the phantom compartments. The relative standard deviations (i.e., the standard deviation over the mean) in the ADC measurements were 0.9%-2.5% for the compartments with larger ADC values (0.9-2.2 μm²/ms), and elevated up to 10.0% for the compartment with smaller ADC values (e.g., 0.3-0.6 μm²/ms). Overall, these results illustrated an excellent reliability of using DW-mSTE-VFA for diffusion parameter quantification in homogeneous diffusion media.

Evaluation of Mango Fruit. FIG. 9 displays a set of representative mango ADC maps estimated using DW-mSTE-VFA and the standard DW-STE sequences, where the diffusion times and b-values of DW-STE acquisition matched those in the DW-mSTE-VFA acquisition. The DW-mSTE-VFA and DW-STE ADC maps showed similar decreasing trend as Δ increased. This observation is further substantiated in Table 1 (first row), where the mean and standard deviation of ADC values from the pulp voxels are listed. The mean ADC value decreased from 0.454/0.458 μm²/ms to 0.261/0.273 μm²/ms as Δ increased from 119.7 to 319.7 ms in the DW-mSTE-VFA/DW-STE sequences. The agreement in ADCs was within 4.4% at all diffusion times between the DW-mSTE-VFA and DW-STE measurements.

TABLE 1

| Δ | Method | 119.7 ms | 219.7 ms | 319.7 ms |
|---|---|---|---|---|
| Mango | DW-mSTE- | 0.454 ± 0.073 | 0.327 ± 0.074 | 0.261 ± 0.077 |
| | VFA | | | |
| | DW-STE | 0.458 ± 0.066 | 0.338 ± 0.070 | 0.273 ± 0.077 |
| Brain WM | DW-mSTE- | 0.810 ± 0.096 | 0.707 ± 0.086 | 0.646 ± 0.081 |
| | VFA | | | |
| | DW-STE | 0.801 ± 0.100 | 0.716 ± 0.109 | 0.657 ± 0.094 |
| Brain GM | DW-mSTE- | 0.702 ± 0.055 | 0.670 ± 0.053 | 0.651 ± 0.056 |
| | VFA | | | |
| | DW-STE | 0.680 ± 0.052 | 0.658 ± 0.048 | 0.664 ± 0.042 |

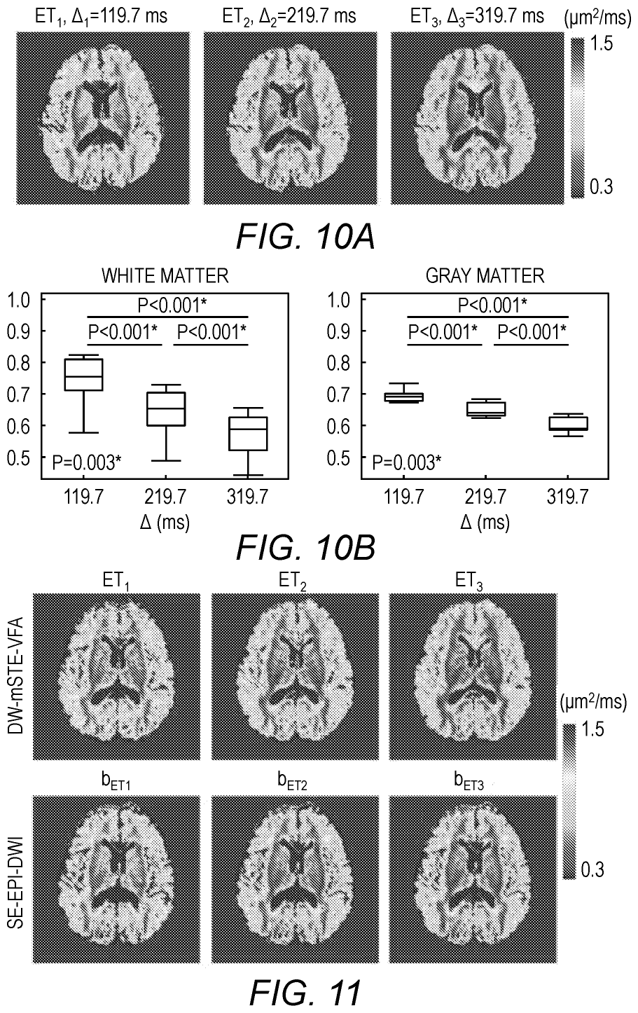

Brain Imaging. FIG. 10A provides a set of representative brain ADC maps obtained from DW-mSTE-VFA at ET₁ (Δ=119.7 ms), ET₂ (Δ=219.7 ms), and ET₃ (Δ=319.7 ms), respectively. Smaller ADC values were observed at longer diffusion time. This trend is quantitatively illustrated in FIG. 10B, where the mean ADC value from the six subjects was box-plotted against diffusion time over the white matter (WM) and gray matter (GM) ROIs, respectively. The p-values generated by Friedman's test are labeled in each boxplot, where significant differences among the ADC values across all three diffusion times were observed in both WM (p=0.003) and GM (p=0.003). The time-dependence is further revealed by the post-hoc paired f-test, where statistically significant differences (p<0.001) were also observed in all diffusion-time pairs for both WM and GM regions.

FIG. 11 shows a comparison of the ADC maps obtained from the DW-mSTE-VFA sequence at ET₁, ET₂, and ET₃, respectively (upper row) versus those obtained from the SE-EPI-DWI sequence at a fixed diffusion time (Δ=37.4 ms) with three sets of b-values (b_{ET1}, b_{ET2}, and b_{ET3}) that matched those used in the DW-mSTE-VFA sequence (lower row). For the DW-mSTE-VFA sequence, the mean ADC values of WM/GM voxels corresponding to ET₁, ET₂, and ETs were 0.751/0.798, 0.693/0.683, and 0.656/0.621 μm²/ms, respectively. For the SE-EPI-DWI sequence, the mean ADC values estimated using b_{ET1}, b_{ET2} and b_{ET3} were 0.839/0.874, 0.783/0.763, and 0.758/0.747 μm²/ms, respectively. The decreasing trend of ADC in the DW-mSTE-VFA sequence as a function of diffusion time outpaced that in the SE-EPI-DWI sequence as a function of b-values.

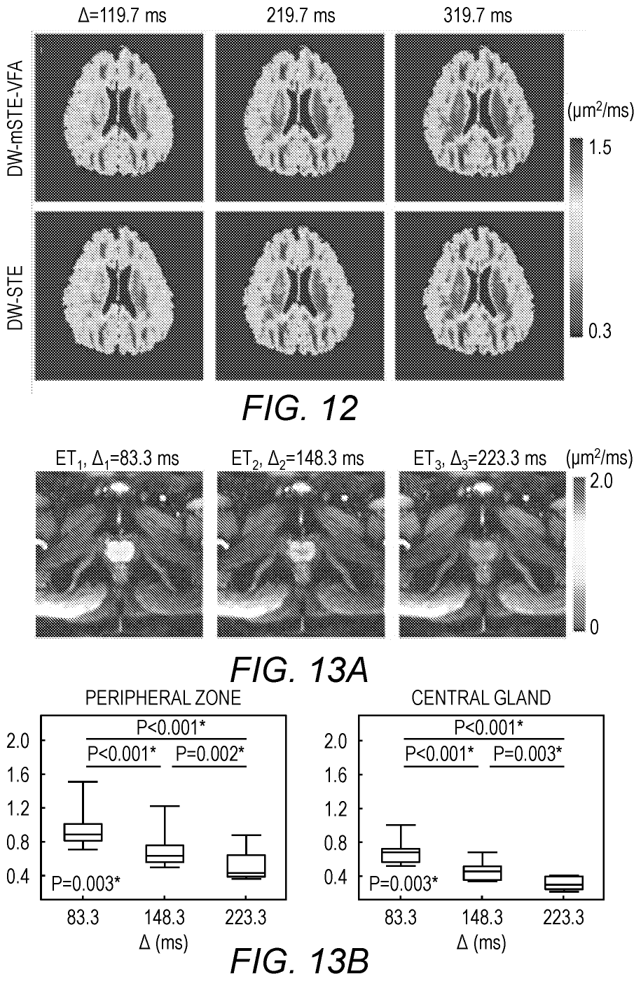

FIG. 12 compares human brain ADC maps obtained from the DW-mSTE-VFA (upper row) and the standard DW-STE sequences (lower row). Similar to the mango experiment, the ADC maps from the DW-mSTE-VFA and DW-STE acquisitions exhibited almost identical trend of decrease as Δ increased. The quantitative results are listed in Table 1 (second and third rows). As Δ increased from 119.7 to 319.7 ms, the mean WM ADC values decreased from 0.810/0.801 μm²/ms to 0.646/0.657 μm²/ms, while the mean GM ADC values decreased from 0.702/0.680 μm²/ms to 0.651/0.664 μm²/ms in the DW-mSTE-VFA/DW-STE acquisitions. The DW-mSTE-VFA and DW-STE measurements exhibited a high degree of agreement with <3.3% difference at all diffusion times. The SNRs measured on the b≃0 s/mm² images in the DW-mSTE-VFA acquisition were 32.1/26.4/22.8 at ET₁/ET₂/ET₃, while the SNRs of the b≃0 s/mm² images in the DW-STE acquisition were 60.5/50.0/46.3 at Δ₁/Δ₂/Δ₃ without signal averaging.

Prostate imaging. FIG. 13A shows the prostate ADC maps overlaid on the raw images (b=0 s/mm²) at ET₁ (Δ=83.3 ms), ET₂ (Δ=148.3 ms), and ET₃ (Δ=223.3 ms) of the DW-mSTE-VFA sequence. Similar to those in the brain imaging study, the ADCs also showed a decreasing trend as the diffusion time increased. Friedman's test showed significant differences among all three diffusion times in both peripheral zone (PZ) (p=0.003) and central gland (CG) (p=0.003), as shown in FIG. 13B. Post-hoc paired t-test revealed statistically significant difference of all diffusion-time pairs in both PZ and CG ROIs (p<0.001 for the 83.3 ms vs. 148.3 ms and 83.3 ms vs. 223.3 ms groups in both the PZ and CG, p=0.002 and 0.003 for the 148.3 ms vs. 223.3 ms group in the PZ and CG, respectively).

What is claimed is:

1. A magnetic resonance imaging system comprising:
a pulse sequence comprising a train of stimulated echoes configured to incorporate a set of multiple b-values and a set of readouts to generate diffusion-weighted images, each corresponding to a b-value in the set of multiple b-values, within a single scan.

2. The magnetic resonance imaging system of claim 1, wherein the pulse sequence comprises a diffusion-weighted preparation module, a spin echo echo-planar-imaging (EPI) module, and a variable flip-angle stimulated echo train EPI module.

3. The magnetic resonance imaging system of claim 1, wherein the stimulated echoes of the pulse sequence comprise an initial 90° excitation radiofrequency (RF) pulse and a subsequent 90° restoration RF, followed by a train of re-excitation RF pulses with variable flip angles (αᵢ), each producing a stimulated echo signal that provides a diffusion-weighted image with its distinctively assigned b-value.

4. The magnetic resonance imaging system of claim 1, wherein one or more crusher gradients or spoiler gradients are used to select a stimulated echo signal corresponding to a specific and desired b-value.

5. The magnetic resonance imaging of claim 3, wherein the variable flip angles (αᵢ) of the train of re-excitation RF pulses are determined recursively by
αᵢ=arctan(sin αᵢ₊₁),
where i=2, . . . n, and n is a total number of re-excitation RF pulses in the train of re-excitation RF pulses and the last flip angle αₙ₊₁ is set at 90°.

6. The magnetic resonance imaging system of claim 1, further comprising a calibration procedure to produce quantitative diffusion coefficient maps by reducing or eliminating the perturbations from relaxation times.

7. A magnetic resonance imaging system comprising:
a pulse sequence comprising a train of stimulated echoes configured to incorporate a set of multiple diffusion times, each corresponding to one diffusion-weighted image in a set of diffusion-weighted images that are collectively acquired in a single scan; the pulse sequence is repeated a plurality of times, each with a unique b-value from a set of b-values.

8. The magnetic resonance imaging system of claim 7, wherein the pulse sequence comprises a diffusion-weighted preparation module and a multiple stimulated echoes with variable flip angles module.

9. The magnetic resonance imaging system of claim 7, wherein the stimulated echoes of the pulse sequence comprises an initial 90° excitation radiofrequency (RF) pulse and a subsequent 90° restoration RF, followed by a train of re-excitation RF pulses with variable flip angles, each producing a stimulated echo signal that provides a diffusion-weighted image with its distinctive diffusion time.

10. The magnetic resonance imaging of claim 9, wherein the variable flip angles (αᵢ) of the train of re-excitation RF pulses are determined recursively by
αᵢ=arctan(sin αᵢ₊₁),
where i=1, . . . n−1, and n is a total number of re-excitation RF pulses in the train of re-excitation RF pulses and the last flip angle αₙ is set at 90°.

11. The magnetic resonance imaging system of claim 7, wherein one or more crusher gradients or spoiler gradients are used to select a stimulated signal corresponding to a specific diffusion time.

12. The magnetic resonance imaging system of claim 7, wherein the diffusion-weighted images with the set of multiple diffusion times and the set of multiple b-values provide the influence of diffusion time on quantification of diffusion-related parameters.

13. The magnetic resonance imaging system of claim 1, wherein the pulse sequence generates datasets from data acquired in a single shot.

14. The magnetic resonance imaging system of claim 7, wherein the pulse sequence generates datasets from data acquired in a single shot.

15. A pulse sequence configured to generate a magnetic resonance image output dataset via a processor, the output dataset comprising temporal diffusion characteristics, the pulse sequence comprising multiple stimulated echoes with variable flip angles.

16. A pulse sequence configured to generate a magnetic resonance image output dataset via a processor, the output dataset comprising different degrees of diffusion-weighting, the pulse sequence comprising multiple stimulated echoes with variable flip angles.

17. The pulse sequence of claim 14, wherein the pulse sequence is configured to obtain the output dataset from a single shot.

18. The pulse sequence of claim 16, wherein the pulse sequence is configured to obtain the output dataset from a single shot.
